(19) United States
(12) Patent Application Publication
(10) Pub. No.: US 2017/0035999 A1
(43) Pub. Date: Feb. 9, 2017

(54) Tissue Expander with Means to Deliver Antibiotics or Medication Uniformly on its Surface Using Multiple Channels Comprising Pores

(71) Applicant: Bandula Wijay, Friendswood, TX (US)

(72) Inventor: Bandula Wijay, Friendswood, TX (US)

(21) Appl. No.: 14/817,968

(22) Filed: Aug. 4, 2015

(51) Int. Cl. A61M 29/02; A61B 90/00

(52) U.S. Cl. CPC A61M 29/02; A61B 90/02; A61M 2205/04

(57) ABSTRACT

A tissue expander distributes antibiotics or other drugs and treats infections to the surrounding tissue. The tissue expander is provided with a manifold that can be accessed from outside via an injection needle which is connected to a series of channels containing drug effusion ports. When the tissue is infected antibiotics are injected into the manifold which in turn runs through the channels, effuse out the strategically placed ports to treat the infection. The procedure can be repeated until the infection is resolved. Any fluids effusing from the tissue are drained using the drainage channels that connect to a central drainage cavity and is later drained out of the body using a needle or cannula placed into a special drainage collection cavity.

### FIELD OF THE INVENTION

The field of the invention comprises methods of delivering antibiotics and other drugs around the external surface of an implanted tissue expander by injection into a chamber on or within the tissue expander that is connected to one or more perforated manifolds with an option of collection and storage of fluid secreted by the surrounding tissue for later removal by a needle or cannula.

### BACKGROUND OF THE INVENTION

Tissue expanders are widely used to increase the volume of a body chamber and thereby increasing the surface area of skin. Tissue expanders are typically sacks made from silicone rubber having specially designed ports that allow the operator to inject fluids, through these ports, into the chamber after placement of the expander within the body chamber. Fluids are injected periodically into the sac in order to gradually increase the volume of the body chamber. In a good number of cases, the tissue expanders get infected after surgical placement. While the tissue expanders are sterile, the periodic and frequent injections as well as the patient's compromised immune system are possible reasons for such infections. When such infections happen, the tissue expander has to be removed. The chamber is washed with anti-microbial solutions, resulting in additional surgery. In some instances the chamber is closed without a tissue expander and thereby needing another surgery after the wound is healed.

In Bark et al. U.S. Pat. No. 5,066,303 a tissue expander is shown with multiple layers out of which innermost and outermost layers are relatively stable (high durometer) materials. The intermediate layer consists of flowable (low durometer) materials in between these outer and inner layers and can be a single-layer or multi-layered, as needed. Unlike previous designs, this design doesn't need a special port or needle entry (septum) since the self-sealing shell of the tissue expander seals by itself when the needle is pulled out.

In Weise U.S. Pat. No. 5,496,368 a self-inflating tissue expander with dual layers is disclosed, which absorbs body fluid into the tissue expander reservoir from surrounding tissue due to osmotic driving force. The patient doesn't feel pain during the fluid intake process into the tissue expander reservoir since there is no piercing through the skin to deliver fluid from an external source and there is no risk of infection during the fluid intake process since there is no penetration of foreign objects, such as needles. In Weise's design, there is no control of fluid intake other than water concentration difference of the inner chamber and that in the outer tissue. It is very hard to control the volume of the tissue expander device in this method. In addition, all devices described in this article do not have a mechanism to control or fight back against complications due to infections.

Rosenberg in U.S. Pat. No. 5,630,843 describes a double chamber tissue expander that comprises two chambers namely infusion solution chamber and expansion solution chamber. Both these chambers are provided with liquid transport means to introduce and remove fluid to and from the chambers. The expansion chamber exerts a pressure on infusion chamber when the expansion fluid is introduced into the expansion chamber after implant of the system. This, or injection into liquid transport means of the infusion chamber causes release of infusion through porous material. The problem with this design is that in the injection mode it is difficult to know how much of the injected antibiotics actually go through the openings and how much of the antibiotics simply inflates the porous member attached to the non-porous inflatable bladder.

In U.S. Pat. No. 8,167,836, Lee et al describes an enclosure wall and drug formulation that includes a drug that is housed within the drug formulation pocket. The pocket contains a solid drug. The drug is released by diffusion and or migration. There is no absolute control as to the rate or the dosage of diffusion or migration of the drug. Additionally the drug cannot be induced intentionally, such as when an infection has evolved and requires immediate treatment. The drug type cannot be changed based on the microbe infecting the tissue and/or patients' tolerance to the type of pain medication when used to treat pain.

U.S. Pat. No. 8,460,383 and U.S. Pat. No. 8,239,057 show multi-compartment inflatable medical devices using a header to connect the compartments. U.S. Pat. No. 7,993,299; U.S. Pat. No. 7,575,565 and U.S. 20120065465 extravasation devices for arthroscopic procedures for draining excess fluid from the patient. U.S. 2012/0046624 and U.S. Pat. No. 9,019,681 show the use of a vacuum pump and manifold for collecting liquid from tissue.

In these and other tissue expander inventions in the past have not dealt with one of the most challenging and inherent problem of infections. Infections pose additional pain and suffering to the patient and result in increased patient care costs due to multiple surgeries and hospital stays.

In addition, in some cases, the implantation of the tissue expander causes severe inflammation and or seroma. Fluid collects around the tissue expander and causes severe pain and edema.

### SUMMARY OF THE INVENTION

The present invention provides the surgeon with a simple and effective way to address the post-surgical medical management the patient occurring when placement of the tissue expander results in infection, inflammation and or seroma. While effort is made to prevent contamination during surgery, such as by using sterile tissue expanders and sterile surgical techniques, a patient's inability to fight infection is not within the surgeon's control. The present invention comprises one or more chambers on or within the outer surface of the tissue expander, into which a drug of choice can be introduced via a needle or similar device or the chamber can be prefilled. The chamber is connected to an array of non-distensible perforated channels that are placed on the surface of the tissue expander in order to deliver antibiotics and other drugs if the tissue surrounding the tissue expander needs medical management such as when there is infection.

In an alternative embodiment, the chamber can be prophylactically pre-filled with antibiotics or a combination of antibiotics. This is a convenient solution to avoid having to puncture the skin to administer the medicine into the tissue expander. However, as the antibiotic to be used could be specific to the type of microbial infecting the tissue and to the patient, not having the chamber prefilled with a specific antibiotic would be the preferred solution to the problem.

The antibiotic chamber can be placed next to or nearby the injection port which is provided for the injection of the expansion fluid. If there is no fluid injection port, the chamber can be located on any part of the tissue expander. Expansion fluid is normal saline and is injected periodically to expand the tissue expander which in turn expands and increases the volume of the body chamber while causing the expansion of the skin surface.

The chamber provided for the injection of the antibiotic can be small sack or space that is placed on or below the surface of the tissue expander, and can be provided with magnetic or other location markers. Alternatively, the sack or space can be placed on or below the surface of any tissue expander, which does not have a fluid injection port such as a mechanical tissue expander that is expanded by a gas contained within it or other expanders that utilize mechanical means or osmotic pressure differential for the purpose of expansion of the tissue expander. The chamber can also be located at a fixed location with a fixed orientation to the fluid injection port or simply at the fluid injection port. The chamber can be placed around the fluid injection port, next to it or completely away from the fluid injection port when its location with respect to the fluid injection port is well marked on the device. The chamber may be connected to a manifold which in turn is connected to multiple non-distensible conduits to carry the medicine to the surface of the tissue expander or, in another design, the non-distensible channels can be directly connected to the drug injection chamber. These channels preferably extend from the top all the way to the bottom of the tissue expander and are preferably placed in a symmetrical or non-symmetrical array. The channels are provided with small holes, or pores, for the medicine to ooze out. These holes can be of different diameters so as to evenly distribute the medication along the length of the channel. The holes near the manifold reservoir could be smaller than the holes further away from the reservoir.

It is also possible to provide two or more separate chambers for drug injection in the tissue expander, with more than one drug injection ports, so that the antibiotic treatment can be limited to a certain area instead of the entire surface contacting tissue.

It is also possible to use the concept of the above described tissue expander to deliver such fluids that would shield the tissue expander materials, which can otherwise deteriorate, from radiation when the patient is treated with radiation such as for cancer treatment.

The bottom of the chamber is provided with a magnetic or non-magnetic metal liner or a high strength plastic liner, such as from Kevlar or polyimide material to prevent the operator piercing through the chamber into the fluid chamber of the Tissue expander. In case of a magnetic metal liner, it will also help the operator to locate the position and orientation of the tissue expander using a device to locate magnetic fields. Tissue expanders are typically provided with magnetic or radiopaque markers that would clearly locate the fluid port. Therefore the port for injection of medicine to the drug chamber will also be provided with appropriate magnetic, non-magnetic or radiopaque markers to differentiate the manifold from the fluid injection port. One aspect of the present invention is that the entire tissue expander and any components thereof can be made from such materials that will not interfere with magnetic resonance imaging. Therefore non-magnetic materials such as nickel titanium alloys, stainless steel 316L, gold or titanium can be the material of choice for the identification of the port area and also can be the material of construction of the puncture proof shield.

The injection port for the antibiotic chamber is provided with a surface and made of a material that self-seals after the injection of the antibiotic or drug.

In some cases the tissue surrounding the tissue expander contains fluid that diffuses from the cellular matrices. This fluid, commonly referred to as seroma needs to be drained from the tissue. A drainage tube is placed at the time of surgery to drain seroma and is withdrawn after a few hours or few days. However the fluid collection continues and often causes significant medical conditions to the patient, including but not limited to inflammation and infection. The present invention deals with this problem by an array of preferably non-distensible drainage channels, similar to the channels for the distribution of antibiotics and drugs. These channels are provided with pores through which the excess fluid enters the channels and flows into a collection chamber. This chamber can be accessed from outside the body via a needle or cannula and the fluid is drained by gravity or by vacuum.

### BRIEF DESCRIPTION OF THE DRAWINGS

FIG. 1 shows a prior art tissue expander having a single port for fluid injection placed between the skin and the muscle in the human body;

FIG. 2 shows the proposed tissue expander with medicine channels and having a separate chamber for drug injection;

FIG. 3 shows the proposed tissue expander having the medicine distribution channels and the injection manifold showing the drug effusion ports;

FIG. 4 shows a top view of the tissue expander identifying the drug injection manifold along with the injection port and the drug distribution channels;

FIG. 5 shows an alternate drug distribution channel arrangement with the second drug manifold placed at the near or at the median of the Tissue expander; and

FIG. 6 shows a detail of the medicine delivery channel which is placed in the inside wall of the tissue expander.

### DETAILED DESCRIPTION OF THE PREFERRED EMBODIMENT

In clinical practice a high percentage of tissue expanders get infected after they are implanted into the tissue. This creates a high risk condition especially to those patients who are immune compromised after cancer treatments. In all cases, the infected tissue expander is surgically removed and the chamber is washed repeatedly before the tissue is either closed or a new tissue expander is implanted. In any case, this causes additional risk to the patient and requires additional surgeries and hospital stays. In other instances, seroma develops after the implantation of the tissue expander especially in breast implantations or the patient incurs severe pain after implantation. The present invention helps to resolve the problem of infection, seroma or pain by injecting antibiotics or pain medications to the tissue when such infection is diagnosed, or the present invention facilitates and allows the fluid to drain when seroma develops. Antibiotics can be injected once or in regular intervals until the infection is resolved. Even different or combinations of antibiotics can be injected to treat such infections. Therefore the present invention helps resolve, alleviate or prevent infections after the tissue expander is implanted. The present invention also provides facility to inject radiation shielding fluids when the patient requires radiation post implantation of a tissue expander.

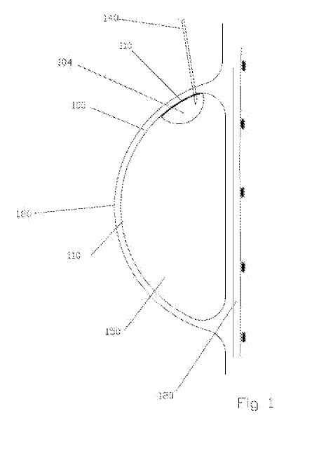

Referring to FIG. 1 the schematic diagram shows a prior art tissue expander. The tissue expander 150 in this case is implanted in the tissue between the skin 160 and the muscle layer 120. The tissue expander essentially consists of an inflatable body 100, which can be inflated by injecting fluids into it. Fluid is injected into the chamber 104 which is connected to the Tissue Expander 150 using a needle 140. The injection port has a self-sealing membrane 110. Once the injection needle 140 is removed, the self-sealing membrane 110 helps to prevent the leakage of fluid out of the tissue expander.

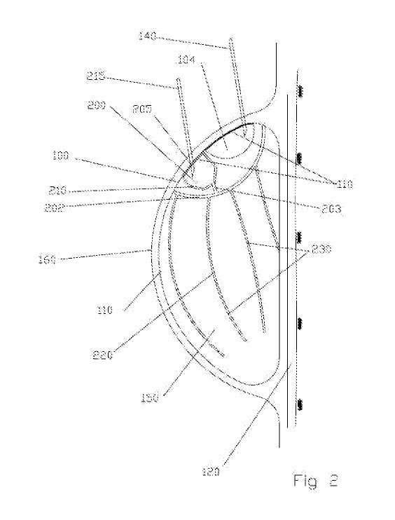

The present invention provides means to inject at one time or periodically, universally or selectively the appropriate antibiotic or other drugs to the tissue either around the entire Tissue expander or to a selected region of the tissue, when the tissue gets infected. When the surrounding tissue is inflamed or in such instances as when the patient is in pain, anti-inflammatory medicines or pain reliving medicines can be injected and distributed to the tissue address such issues. To achieve this objective, as shown in FIG. 2, the present invention provides a chamber 200 into which antibiotic or other drugs can be injected using a needle or cannula 215 which enters the body at the injection port 205 by piercing the skin. The chamber 200 is connected to an injection port 205 dedicated to the injection of drugs, and the injection port is electromagnetically identifiable. The chamber 200 is connected to a manifold 202 via a conduit 203. The antibiotic or other fluids are injected into the chamber 200. Such fluids are then delivered all around the tissue expander and into the tissue through channels 220 that radiate from the antibiotic delivery manifold 202. The manifold 202 provides a convenient way to evenly distribute the drugs or fluids to the plurality of channels 220. FIG. 2 is a schematic representation and conceptual design of the proposed system showing the different components of such a system. While other variations of the design are possible, the intention of the schematic is to describe the principle of operation.

The drug injection port 205 is provided with a self-sealing membrane 110′ and the chamber 200 is provided with a metal plate 210 to prevent the injection syringe puncturing the chamber and entering the tissue expander fluid space. The plate 210 can be made from thin magnetic or non-magnetic materials like stainless steel 316L, titanium or from a variety of metals.

The drug injection port 205 is a part of the chamber 200 along with the puncture proof shield 210. For the delivery of the drugs to the rest of the tissue expander non-distensible surface channels 220 radiate from the manifold 202 to other areas of its surface. These channels 220 can be on located on the surface of the tissue expander, embedded in its wall or be placed in the inner wall of the tissue expander, and have the same purpose for transporting antibiotics or other drugs to infected tissue. FIG. 2 also shows the pores 230 that are placed systematically on the channels 220. One or both ends of the channels 220 can be closed depending on the connection location of the channels 220 to the manifold 202. The pores 230 can be of same or different sizes. In larger tissue expanders, it is advantageous to have pore sizes of increasing diameter placed on the channels, with larger diameter been placed away from the antibiotic delivery chamber 200.

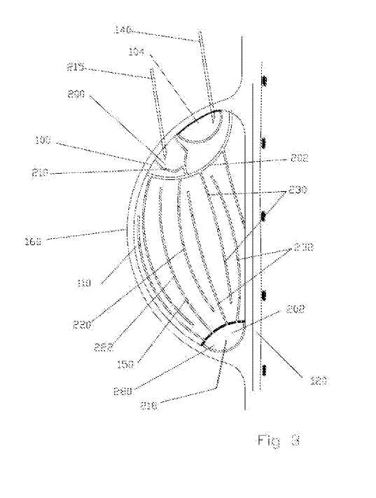

FIG. 3 is a lateral view of the tissue expander showing the chamber 200 for the antibiotic delivery along with several channels 220 which will transport the antibiotics and/or other fluids to the various parts of the tissue expander's surface and thereby to the surrounding tissue. This version of the tissue expander is provided with additional channels for drainage of fluids from the surrounding tissue. The non-distensible drainage channels 222 drain the fluids into a collection chamber 280. The collection chamber 280 is similar to the drug injection chamber 200 in that it also has a self-sealing surface and a puncture proof base shield. The collection chamber 280 can be accessed via a needle or cannula 218 by piercing the skin in order to drain the fluids due to seroma either by gravity or by applying vacuum.

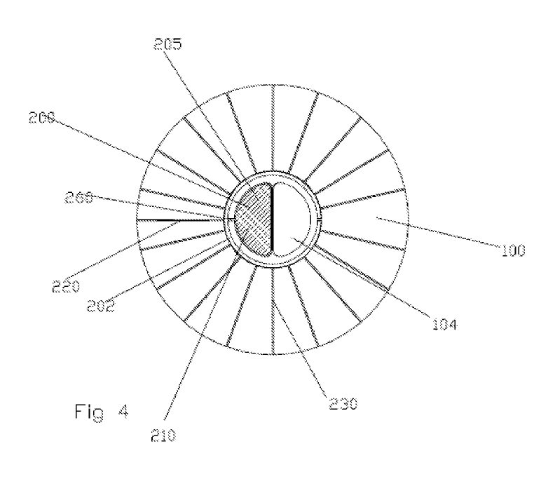

FIG. 4 is top view of the proposed tissue expander. It shows the fluid injection port 130 for the injection of fluids for the inflation of the tissue expander and the antibiotic injection port 205 leading to the chamber 200. In another variation of the same invention, one can combine (not shown) the drug injection chamber 205 with the drug delivery manifold 202 without deviating from the essence of this invention. The metal shield 210 (cross hatched) is placed within the drug injection chamber 200 to prevent the needle from penetrating the manifold wall. The metal shield can be magnetic or non-magnetic. The metal shield 210 can be contoured to the shape of the manifold or the drug injection chamber 200 and depending on the chamber design can be contoured in order to prevent the needle accidentally entering the fluid injection chamber 104′.

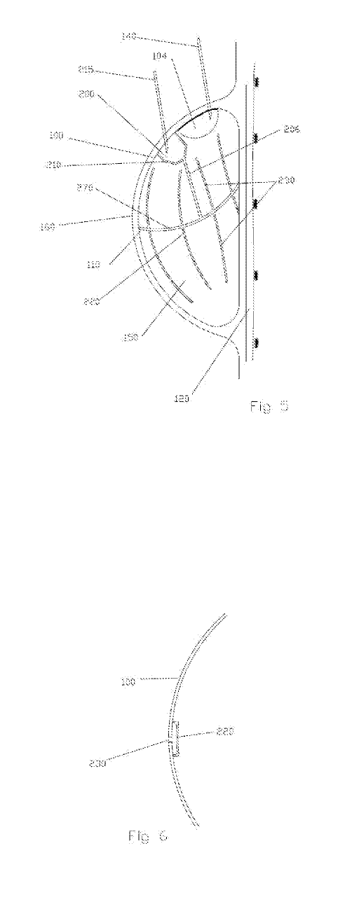

FIG. 5 shows an alternate arrangement of the manifold and the distribution channels for the antibiotics and other drugs or fluids. In this case the drug injection chamber 200 is connected via conduit 206 to manifold 270 which placed near the median of the tissue expander which in turn is connected to drug distribution channels 220. This design will be more useful for larger tissue expanders. In this design an even drug distribution can be achieved due to the placement of the manifold at the median line of the tissue expander.

FIG. 6 shows the arrangement of the drug distribution manifold within the tissue expander. These channels can be placed on the outer surface, within the wall or in inner surface of the tissue expander wall 100′. By placing the drug distribution channels in the inside wall 100′, the outer surface of the tissue expander are smooth or textured, which helps during retrieval of the tissue expander. FIG. 6 shows the drug distribution channel 220 placed in the inside wall of the tissue expander wall 100′ with drug effusion ports 230 placed on the channels 220 and shall be evenly or strategically placed along the channel 220.

It should be noted that the non-distensible nature of the channels 220 ensures that the volume of drugs delivered actually exits the openings 230 without impacting the degree of inflation of the tissue expander. The drainage channels 222 can either be nested with channels 220 as shown in FIG. 3 or they can be spaced apart as much as possible by placement on opposed sides of the tissue expander. This minimizes any tendency for injected drugs into channels 220 taking a short cut into drain channels 222 and instead being absorbed into the surrounding tissue.

The above description is illustrative of the preferred embodiment and many modifications may be made by those skilled in the art without departing from the invention whose scope is to be determined from the literal and equivalent scope of the claims below:

I claim:

1. A tissue expander for expansion of tissue of a patient, comprising:
an expandable body;
a non-distensible drug distribution network on said body comprising a fluid injection area for access through the tissue of the patient to allow fluid delivery into said distribution network without material volume change to said body.

2. The expander of claim 1, wherein:
said expandable body comprising a non-porous compartment with an inflation fluid injection area for access through the tissue of the patient wherein fluid delivery to said distribution network does not materially change an inflated state of said compartment;
said drug distribution network comprises a plurality of spaced injection conduits.

3. The expander of claim 2, wherein:
said injection conduits are disposed on at least one of an exterior surface of said body, a body wall that defines said non-porous compartment and within said compartment with at least one outlet extending through said wall.

4. The expander of claim 2, wherein:
said conduits are interconnected with a manifold.

5. The expander of claim 4, wherein:
said manifold is disposed at one end of said conduits or between opposed end of said conduits.

6. The expander of claim 2, wherein:
said conduits comprise openings along their length with said openings being the same or different sizes.

7. The expander of claim 5, wherein:
at least one end of said conduits is closed.

8. The expander of claim 5, wherein:
said fluid injection area defines a part of at least one injection chamber fluidly connected to said manifold and accessed through the tissue of the patient with a needle or cannula.

9. The expander of claim 8, wherein:
said injection chamber comprises a magnetic or non-magnetic shield to resist penetration of a needle or cannula into said adjacent non-porous compartment.

10. The expander of claim 2, further comprising:
spaced drainage conduits on said body leading to a drainage collection volume on said body, said drainage collection volume comprising a drainage area while isolated from said non-porous compartment, said drainage collection volume for collection of seroma from the tissue of the patient for a predetermined time until removal through said drainage area with a needle or cannula.

11. The expander of claim 10, wherein:
said injection and drainage conduits are substantially parallel.

12. The expander of claim 11, wherein:
said drainage and injection conduits are disposed in a nested pattern or on spaced locations from each other on said body.

13. The expander of claim 8, wherein:
said at least one chamber comprises multiple chambers connected to discrete said injection conduits.

14. The expander of claim 13, wherein:
at least one of said multiple chambers is prefilled with a drug before surgical placement.

15. A tissue expander for expansion of tissue of a patient, comprising:
an expandable body comprising a non-porous compartment;
spaced drainage conduits on said body leading to a drainage collection volume on said body, said drainage collection volume comprising a drainage area while isolated from said non-porous compartment, said drainage collection volume for collection of seroma from the tissue of the patient for a predetermined time until removal through said drainage area with a needle or cannula.

16. The expander of claim 15, wherein:
said drainage conduits are substantially parallel and further comprise openings along their length.

17. The expander of claim 15, wherein:
said drainage collection volume eliminating the use of a drain tube when the expander is surgically placed in the patient.

18. The expander of claim 15, further comprising:
a non-distensible drug distribution network on said body comprising a drug injection area for access through the tissue of the patient to allow drug delivery into said distribution network without material change to an expanded state of said compartment;
said drug distribution network comprises a plurality of spaced injection conduits;
said drainage conduits are nested with said injection conduits or said drainage conduits are grouped together in a spaced relation to said injection conduits.

19. The expander of claim 15, wherein:
said drainage conduits are disposed on at least one of an exterior surface of said body, a body wall that defines said non-porous compartment and within said compartment with at least one outlet extending through said wall.

20. The expander of claim 3, wherein:
said body comprises a smooth exterior surface or an exterior surface with ridges formed by said injection conduits.

21. The expander of claim 19, wherein:
said body comprises a smooth exterior surface or an exterior surface with ridges formed by said drainage conduits.

22. The expander of claim 15, wherein:
said expandable body comprising an inflation fluid injection area for access through the tissue of the patient.

23. The expander of claim 1, wherein:
said body is expanded with fluid pressure, mechanically or with an osmotic driving force.

24. The expander of claim 15, wherein:
said body is expanded with fluid pressure, mechanically or with an osmotic driving force.
